(19) United States
(12) Patent Application Publication
(10) Pub. No.: US 2006/0166367 A1
(43) Pub. Date: Jul. 27, 2006

(54) Protein assay method and test device

(75) Inventors: Paul Satoh, Holt, MI (US); James L. Herbert, Lansing, MI (US); Brinton M. Miller, Narragansett, RI (US)

Correspondence Address: Ian C. McLeod;McLeod & Moyne, P.C., 2190 Commons Parkway, Okemos, MI, 48864, US

(73) Assignee: Neogen Corporation, Lansing, MI (US)

(21) Appl. No.: 11/044,147

(22) Filed: Jan. 27, 2005

(51) Int. Cl. G01N 33/50

(52) U.S. Cl. 436/86

(57) ABSTRACT

A test device for detecting the presence of protein or other reducing compound wherein a bicinchoninic acid or salt composition is in a sampling swab (27) on a sampling wand (17) is separate from a dried cupric salt on a disc (48) in an essentially transparent well (45). The reaction of protein with the cupric salt in the well in the presence of the composition reduces Cu⁺² to Cu⁺¹ which forms a deep purple complex with the bicinchoninic acid or salt and which can be detected visually or by a spectophotometric device (10) or other detection instruments. The result determines whether or not surface is contaminated with protein or other reducing compound and thus is unsanitary.

### CROSS-REFERENCE TO RELATED APPLICATIONS

Not Applicable.

### STATEMENT REGARDING FEDERALLY SPONSORED RESEARCH OR DEVELOPMENT

Not Applicable

### STATEMENT REGARDING GOVERNMENT RIGHTS

Not Applicable

### BACKGROUND OF THE INVENTION

(1) Field of the Invention

The present invention relates to an improved protein assay method and test device which utilizes bicinchoninic acid or a salt as a compound in a contained solution in a wand with a swab for sampling a surface for the presence of protein as a contaminant. A cupric salt is provided in a well of a container for the wand to produce a purple color change in the presence of protein on a surface when the solution is flushed through the sampling swab. The cupric salt is dried in the container of the test device. The protein is washed from the sampling swab by breaking a seal on the wand between the solution and the sampling swab. The test device particularly prevents the reduction of Cu⁺² to Cu⁺¹ over time prior to use of the test device due to the separation of the cupric salt in the container from the compound in the wand.

(2) Description of the Related Art

U.S. Pat. No. 4,839,295 to Smith describes the reaction of bicinchoninic acid and a cupric salt at basic pH in the presence of a protein. U.S. Pat. No. 5,726,062 to Numa et al describes the use of a bicinchoninic reagent which is a solution of a mixture of a cupric salt and a bicinchoninic acid or salt in a sampler swab. The problem with this construction is that the Cu⁺² degrades over time to Cu⁺¹ in the presence of the bicinchoninic acid or salt.

### OBJECTS

A test device for light production is described in U.S. Pat. Nos. 6,541,194, 6,548,018 and 6,653,147 to DeCesare et al. These devices require a luminometer to detect chemiluminescence produced in the presence of bacterial contamination. There is no suggestion of a spectrophotometer.

A problem also arises because of the use of organic polymers for a swab for surfaces because of the presence of groups integrated into the surfaces of the organic polymers which reduce Cu⁺² to Cu⁺¹. There was thus a need for an improvement in the devices of the prior art.

It is an object of the present invention to provide a test device which maintains the cupric salt separate from the bicinchoninic acid or salt compound until use in dry form. It is also an object to provide a test device which is economical to construct and reliable.

It is further an object of the present invention to provide a novel test device which prevents unwanted reduction of Cu⁺² to Cu⁺¹ prior to use of the device. These and other objects will become increasingly apparent from the following description and the drawings. These and other objects will become increasingly apparent by reference to the following description.

### SUMMARY OF THE INVENTION

The present invention relates to a test device comprising:

(a) a sampling wand for an assay for the presence of protein or other reducing compound on a surface, the wand comprising:
(i) an internal reservoir disposed toward a distal end of the wand;
(ii) a bicinchoninic acid or alkali metal salt compound in solution within the internal reservoir;
(iii) an external sampling swab disposed on a surface at the distal end of the wand for picking up protein on the surface; and
(iv) a frangible seal disposed between the sampling swab and the internal reservoir, so that when the seal is broken the solution containing the compound rinses through and from the swab to release any of the protein or other reducing compound (such as a sugar) from the swab; and

(b) a container for inserting the wand and for receiving the released solution from the wand so that in the presence of any of the protein or other reducing compound a cupric ion from a dried inorganic cupric salt in the container is reduced to a cuprous ion by the protein or reducing compound and reacts with the compound to produce a color indicating the presence of the protein or reducing sugar.

The present invention also relates to the test device wherein the cupric salt is dried in the presence of a non-ionic surfactant on a porous member in the container. The test device wherein the cupric salt is cupric sulfate pentahydrate dried with a non-ionic surfactant on a porous member, which is comprised of a silicone elastomer, in a well in a bottom portion of the container. The test device wherein the porous member is a polyvinyl alcohol. The test device wherein the cupric sulfate pentahydrate was provided by a 1% by weight to volume aqueous solution of the cupric sulfate pentahydrate with 0.05% by volume non-ionic surfactant which is dried on the porous member. The test device wherein the non-ionic surfactant is TRITON X-100™ (polyethylene glycol tert-octylphenyl ether). The test device wherein the sampling swab on the wand is comprised of a porous, absorbent material. The test device wherein the sampling swab has a cylindrical shape. The test device wherein the sampling swab has a height which is less than a diameter and is comprised of a porous silicone elastomer. The test device further comprising a non-ionic surfactant as an extracting agent absorbed in the sampling swab. The test device of wherein the non-ionic surfactant is TRITON X-100™. The test device wherein the solution in the wand is a buffered solution at a basic pH. The solution with basic pH is enclosed in a capsule made of polyolefin and sealed with “sealable” oriented polyolefin film (heat seal ˜95° C. for ˜10 seconds.)

The test device wherein the composition is a sodium salt and has a concentration of 0.01 to 1% (wt/vol) in the solution in the wand which is buffered with citrate and tartarate buffer and is at a pH of about 11. The sampling wand wherein the solution in the wand comprises a buffer to dissolve compound and is at a basic pH. The test device wherein a puncturing means for the frangible seal is provided in the container so that when the wand is inserted into the container the frangible seal is punctured by a piercing member to release the solution from the wand, through the swab and into the container with the cupric salt. The test device wherein the swab and the frangible seal are both punctured.

The present invention also relates to a method for detecting a protein or reducing sugar on a surface which comprises:

(a) providing a test device comprising:
(i) a sampling wand for an assay for the presence of protein on a surface, the wand comprising:
(ii) an internal reservoir disposed toward a distal end of the wand;
(iii) a bicinchoninic acid or alkali metal salt compound in solution within the internal reservoir;
(iv) an external sampling swab disposed on a surface at the distal end of the wand for picking up protein on the surface; and
(iv) a frangible seal disposed between the sampling swab and the internal reservoir, so that when the seal is broken the solution containing the compound rinses through and from the swab to release any protein from the swab; and a container for inserting the wand and for receiving the solution from the wand so that in the presence of protein a cupric ion from a dried inorganic cupric salt in the container is reduced to a cuprous ion by the protein or other reducing compound and reacts with the compound to produce a color indicating the presence of the protein or other reducing compound;

(b) wiping the sampling swab on the wand over a surface to pick up any protein or reducing compound;

(c) inserting the wand in the container and breaking the frangible seal so that the released solution washes the swab contacts the inorganic cupric salt and any protein or other reducing compound reduces the cupric ion to the cuprous ion and reacts with the compound (BCA); and

(d) determining whether or not the protein or other reducing compound was present on the surface, wherein green is negative and purple is positive for protein or reducing compound.

The present invention also relates to the method wherein the test device is provided with a puncturing means for the frangible seal in the container and wherein the wand is inserted into the container, the frangible seal is broken by the puncturing means to release the solution from the wand through the swab and into the container with the cupric salt. In the preferred method both the frangible seal and the swab are punctured. In the preferred method the cupric salt is dried with a non-ionic surfactant on a porous member in the container. In the preferred method the cupric salt is cupric sulfate hexahydrate dried with a non-ionic surfactant on a porous member, which is comprised of a silicone elastomer, in a well in a bottom portion of the container. The method wherein the porous member is a polyvinyl alcohol or a silicone polymer. In the preferred method the cupric sulfate pentahydrate was provided by a 1% by weight to volume aqueous solution of the cupric sulfate pentahydrate and a 0.05% by volume non-ionic surfactant which is dried on the porous member. In the preferred method the non-ionic surfactant is TRITON X-100™ (polyethylene glycol tert-octylphenyl ether). In the preferred method the sampling swab on the wand is comprised of a porous, absorbent material. Preferably the sampling swab has a cylindrical shape. Preferably the sampling swab has a height which is less than a diameter and is comprised of a silicone elastomer. Preferably a non-ionic surfactant as an extracting agent is absorbed in the sampling swab. Preferably the non-ionic surfactant is TRITON X-100. Preferably the solution in the wand is a buffered solution at a basic pH. Preferably the compound is as a sodium salt and has a concentration of 0.01-1% (wt/vol) in the solution in the wand which is buffered with citrate and tartarate and is at a pH of about 12. Preferably the solution in the wand comprises an acid neutralizing agent and is at a basic pH.

### BRIEF DESCRIPTION OF THE DRAWINGS

FIG. 1 is a perspective view of an assay device 10 optionally used in the present invention.

FIG. 2 is a perspective view of the sampling wand 17 of the device 10 of FIG. 1.

FIG. 3 is a perspective view of the analysis structure or container 30 of the device of FIG. 1.

FIG. 4 is an exploded view of the sampling/analysis member 15 of the device of FIG. 1.

FIG. 5 is a cross-sectional view of the sampling/analysis member 15 of the device 10 of FIG. 1.

FIG. 6 is an illustration of the sampling wand 17 of the device of FIG. 1 sampling a surface 60 suspected of bacterial contamination.

FIG. 7 is a cross-sectional view of the sampling/analysis member 15 of the device of FIG. 1 illustrating the sampling wand 17 in a first operative position within the sampling/analysis device.

FIG. 8 is a cross-sectional view of the sampling/analysis member 15 of the device of FIG. 1 illustrating the sampling wand 17 in a second operative position within the sampling/analysis device.

FIG. 9 is a magnified view of the distal end of the sampling/analysis member 15 of the device of FIG. 1 with the sampling wand 17 in the second operative position within the sampling/analysis device.

FIG. 10 is a partial cut-away perspective view of the spectrophotometer 10 of the device of FIG. 1.

### DETAILED DESCRIPTION OF THE INVENTION

In general, the present invention provides an apparatus and methods that make possible the rapid detection through a color reaction of materials indicative of the presence of protein on a surface. The present invention is capable of use by unskilled operators under the relatively harsh field environment of institutional food preparation services, health care providers and the like. The results are easily detected visually, but can also be determined spectophotometrically by the device 10.

Turning now to the Figures, there is provided in FIG. 1, an illustration of a preferred hand-held automatic spectophotometric assay device of the present invention, shown generally at 10. Shown in FIG. 1 is the sampling/analysis member 15, and the hand-held spectrophotometer 20, designed to accept the sampling/analysis member 15. As can be seen from FIG. 1, and the following Figures, the spectrophotometer 20 of the present invention is of a scale that can easily fit into an operator's hand, making possible essentially single-handed operation. The sampling/analysis member 15 can be held in one hand and easily inserted in the sample port 24 of the spectrophotometer as the operator holds the device in the operator's other hand. Once the internal electronics of the spectrophotometer 20 are in a ready state, full insertion of the sampling wand 17 into the assembly already inserted into the spectrophotometer brings the bicinchoninic reaction into close proximity to conventional spectophotometers detector circuitry (not shown). A digital readout is then displayed on the display screen 28. The readout displayed on the screen informs the operator of the relative hygienity of the sampled surface based upon the detection of a purple color indicating the presence of protein. The color can also be detected visually or by digital photographic means.

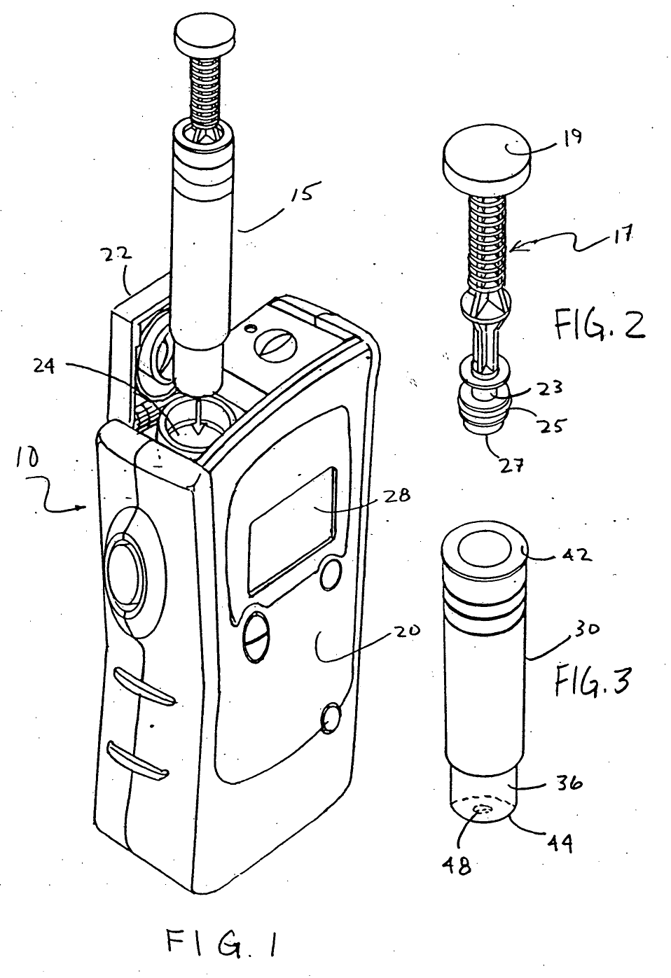

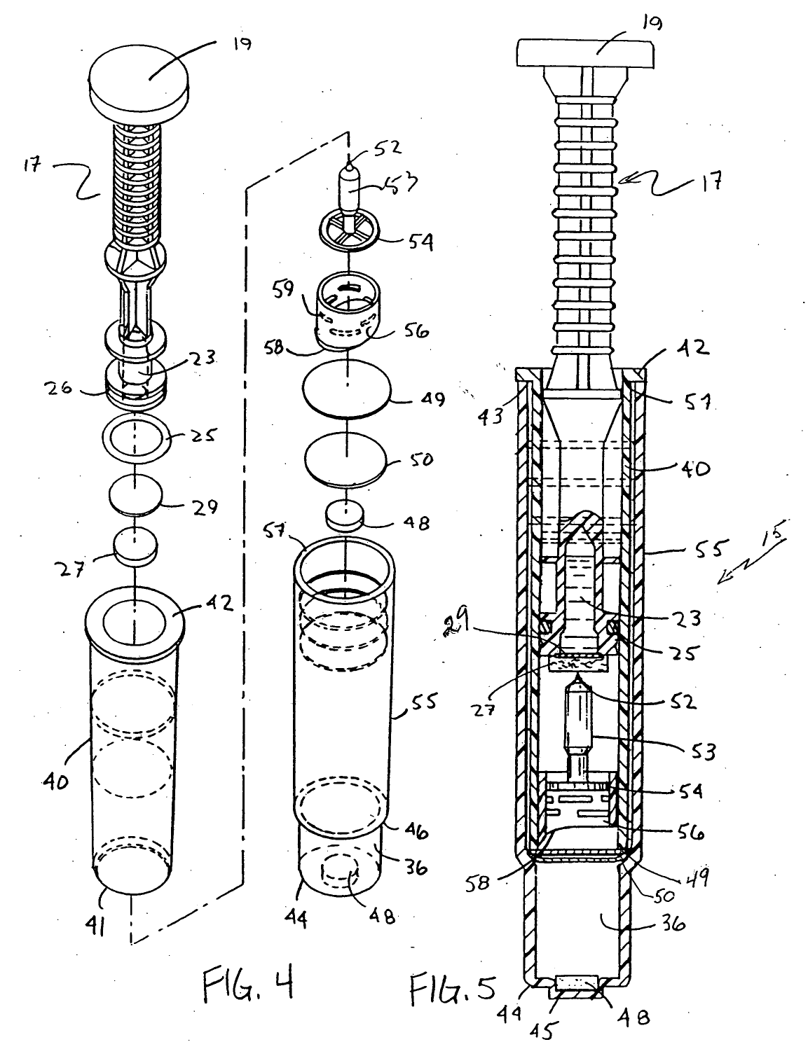

By reference to FIGS. 2 and 3, the sampling/analysis member 15 is depicted to illustrate two of the component structures of the member. FIG. 2 illustrates the sampling wand component 17 of the sampling/analysis member 15. The sampling wand 17 is further comprised of a top 19 located at the proximal end of the wand. The primary purpose of the top 19 is to provide a structure that facilitates the operator's manipulation of the sampling wand 17 as the wand is moved between specific positions within the inner chamber (not illustrated) of the sampling/analysis member 15. Although FIG. 2 illustrates the top 19 in a substantially flat cylindrical shape, it will be appreciated that this shape is for illustrative purposes only, and that other, equally useful, geometries are possible and within the grasp of one of ordinary skill in the appropriate art.

Also illustrated in FIG. 2 are additional structural elements of which the sampling wand 17 is comprised. These include a reservoir 23 located toward the distal end of the sampling wand 17. This reservoir is of approximately 200-250 μL in total volume. As will be discussed in greater detail below, the contents of this reservoir that, in one embodiment, comprise a bicinchoninic acid or salt compound in solution are released into the inner chamber (not illustrated) of the sampling/analysis member by piercing structures located within that inner chamber. The sampling wand 17 further comprises a swab disc 27 adhered to the exterior of the distal end of the sampling wand 17, and on a common vertical axis with the wand. Also illustrated in FIG. 2 is an o-ring structure 25 located toward the distal end of the wand 17, and situated on the exterior of the cylindrically shaped distal portion. The purpose of the o-ring 25 is to provide a sealing fit between the outer surface of the distal portion of the wand 17 and the inner surface of the inner chamber (not illustrated) of the sampling/analysis member 15, as the wand 17 moves longitudinally through the inner chamber. It is preferred to achieve such sealing fit between the wand 17 and the inner chamber in order to prevent the drying out of the pre-wetted sampling swab 17 (disc 27).

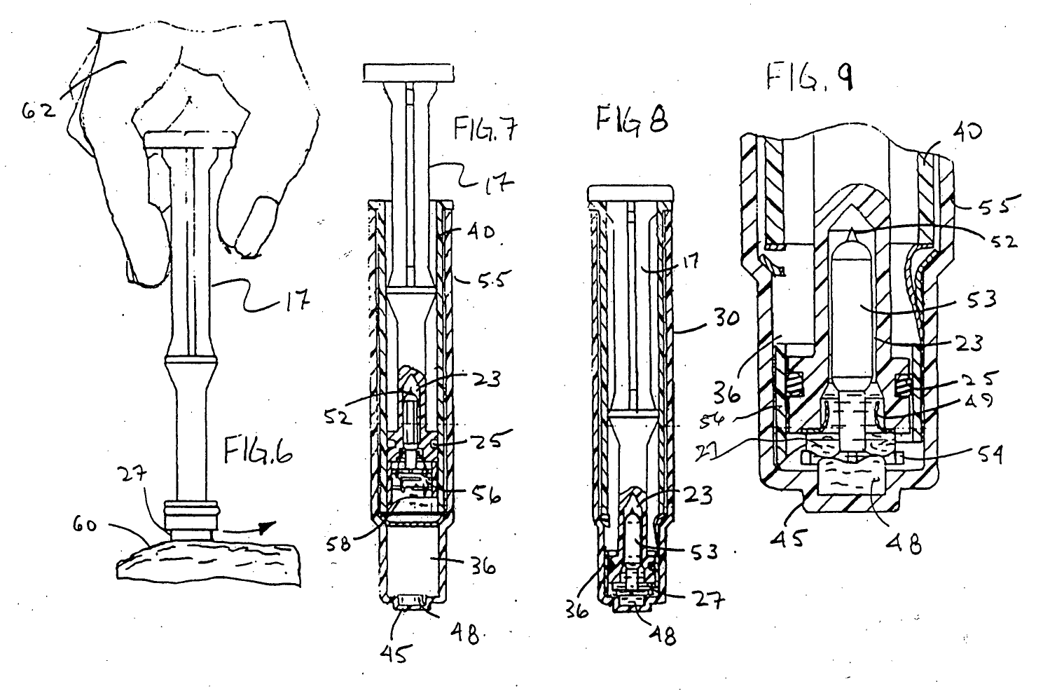

Turning to FIG. 3, there is illustrated the analysis structure 30 of the sampling/analysis member 15 of the device of the present invention. The analysis structure 30 is substantially cylindrical in shape and is actually comprised, in the embodiment illustrated in FIG. 4, of two separate but mating components, an inner chamber 40, and an outer chamber 55. As will be recognized by one of skilled in the appropriate art, the use of two separate structures in the sampling/analysis member is dictated more by manufacturing concerns than by operational factors and that the present invention contemplates a device that may be constructed of a single chamber. Located at the distal end of the outer chamber 55 of the analysis structure 30 is a well 36 that, as is apparent from FIGS. 3-5, is co-linear along the same central axis as the inner chamber 40 and the analysis structure 30. The diameter of the cylindrically shaped reaction well 36 is slightly smaller than the diameter of the outer chamber 55. The point of juncture between the walls of the outer chamber 55 and the slightly narrower walls defining the reaction well 36 portion of the outer chamber form a shoulder region 46, best seen in FIGS. 4 and 5. In the bottom wall 48 of the reaction well 36 is a disc cavity 45, best seen in FIG. 5, and seen in hidden lines in FIG. 3. The disc cavity 45 holds the disc 48 with the inorganic cupric salt, the composition of which is discussed in more detail below. Also illustrated in FIG. 3 is the top rim 42 of the inner chamber 40. As is best illustrated in FIGS. 4 and 5, the bottom edge 43 of the top rim 42 of the inner chamber, in the fully assembled arrangement of the sampling/analysis member 15, rests on the top edge 57 of the outer chamber 55.

FIG. 4 illustrates the sampling/analysis member 15 of the device of the present invention in an exploded view. Part numbers are consistent with the part numbers referenced in FIGS. 1-3 for identical structural elements, a convention adhered to throughout this description. Starting from the top, or proximal, end of the sampling/analysis member, there is shown a top 19 of the sampling wand 17. Toward the distal end of the sampling wand 17, there is shown the reservoir 23, and the o-ring channel 26. Immediately below the distal end of the sampling wand 17, there is shown the o-ring 25 that sits in the o-ring channel 26 to provide, as discussed above, a sealing fit between the sampling wand 17 and the inner walls of the inner chamber 40. Shown immediately below the o-ring is the upper seal 29 that sits on the lower edge/surface (not shown) of the distal end of the sampling wand 17. The seal 29 is made of a frangible material, preferably an impervious polymeric film, and is adhered through use of an appropriate adhesive to the bottom edge/surface of the sampling wand. The seal 29 serves to seal the bicinchoninic acid or salt solution within the reservoir 23, and to prevent the diffusion of species from the reservoir across the membrane. The next component illustrated in FIG. 4 is the sampling swab 27, the composition of which is discussed in more detail below. The sampling swab 27 is affixed to the bottom of the sampling wand 17, with the upper seal 29 interposed between it and the reagent reservoir 23.

The next component illustrated FIG. 4 is the inner chamber 40 of the sampling/analysis member 15. The inner chamber 40 is cylindrical in shape and sized to fit snugly within the outer chamber 55, also shown in FIG. 4. Located at the proximal end of the inner chamber 40 is the top rim 42. At the distal end of the inner chamber is the bottom edge 41. As can be seen in FIG. 4, the cylindrically shaped inner chamber 40 is open at both ends. The top, or proximal, end of the inner chamber is effectively closed by the bottom of the sampling wand 17, through the sealing effect of the o-ring 25 as it contacts the inner walls of the inner chamber 40. The bottom, or distal, end of the inner chamber 40 is sealed by a first seal 49, as seen on the right side of FIG. 4, affixed with an appropriate adhesive to the bottom edge 41 of the inner chamber. Like the upper seal, the first seal 49 is composed of a frangible material.

Moving to the right side of FIG. 4, there is shown, at 53, a piercing member, comprised of a circular base 54 at the distal end, and a point 52, at the proximal end. Immediately below the piercing member 53 is the cutting member 56, which is substantially cylindrical in shape. Each end of the cutting member 56 is open, and the distal, or bottom, edge of the cutting member, the cutting edge 58, is angled so that the top edge is not parallel to the cutting edge. Also illustrated is the base channel 59 that is circumferentially positioned within the cutting member 56. The base 54 of the piercing member 53 rests within the base channel 59 so that, when the sampling/analysis member 15 is fully assembled, as is illustrated in FIG. 5, the piercing member 53 sits within the cutting member 56, which, in turn, sits within the inner chamber 40 of the sampling/analysis member 15. The central axis of the piercing member 53 is co-extensive with the central axis of the cutting member 56, the inner chamber 40, and the outer chamber 55 of the sampling/analysis member 15.

Second seal 50 is affixed through the use of an appropriate adhesive to the shoulder region 46 of the outer chamber 55. However, in an alternative embodiment of the device, the outer chamber can be constructed without the second seal 50; however, this is not preferred since the dried reagents must be protected from moisture. Manufacturing concerns, rather than operational concerns, will frequently dictate the use of both first 49 and second 50 seals. The final component of the sampling/analysis member 15 illustrated in FIG. 4 is the disc 48. As is illustrated by the hidden lines at the distal end of the outer chamber 55, the disc 48 sits within a reagent disc cavity 45 (best seen by reference to FIG. 5) in the bottom of the reaction well 36.

FIG. 5 provides a cross-sectional view of the fully assembled sampling/analysis member 15 prior to use. By reference to FIG. 5, it will be possible to gain an appreciation of the relative positioning of the individual components of the member 15 in this assembled state. In this state, the bottom edge 41 of the inner chamber 40 rests on the shoulder region 46 of the outer chamber 55, toward the distal end of that chamber. Also apparent are the first 49 and second 50 seals positioned on the bottom edge 41 of the inner chamber and the shoulder region 46 of the outer chamber, respectively. By reference to FIG. 5, it can be seen that the cutting member 56 is positioned within the inner chamber 40 so that the distal cutting edge 58 is positioned directly above the first and second seals, 49 and 50. In the assembled state, the piercing member 53 sits with its base 54 situated within the cutting member 56, specifically within the base channel 59 of the cutting member. In the fully assembled arrangement, the point 52 of the piercing member 53 is positioned immediately below the sampling swab 27. Immediately above the sampling swab 27, on the proximal side of the upper seal 29, is the reagent reservoir 23 in the sampling wand 17. As provided in the assembled configuration, the sampling/analysis member 15 may be provided with an external seal (not shown) that serves as a vapor barrier preventing loss of reagent or wetting solution from within the device.

Referring now to FIGS. 6 through 9, there is illustrated the sequential operation of the sampling/analysis member of the device of the present invention. FIG. 6 illustrates the use of the sampling wand 17, held in a single hand 62 of the operator, to obtain a sample from a surface 60 suspected of protein contamination. In a preferred manner, the sampling/analysis member 15 is first inserted into the port 24 of the assay device 10. If a protective external seal has been provided with the sampling/analysis member, then the seal must first be broken and/or removed before the sampling wand 17 can be inserted into the port 24. As can be seen from FIG. 6, the sampling wand 17 is then removed from the inner chamber 40 of the sampling/analysis member 15. Once removed, the sampling wand 17 can be placed in close proximity to the surface to be sampled so that the sampling swab 27 contacts the surface. As will be discussed in more detail below, the sampling swab 27 is preferably packaged and sealed in the sampling/analysis member in a pre-wetted state. More preferably, the sampling swab 27 is pre-wetted with a solution of an extracting agent, preferably in an appropriate buffer to maintain the solution at a pH value in the range of 5.7 to 7.5. A preferred extracting agent is non-ionic surfactant.

Several suitable surfactants or combination of surfactants are known to those skilled in the art and include non-ionic detergents such as TRITON X-100™, TWEEN 20 and TWEEN 80. The concentration of surfactant solution varies for each type of detergent and can range from 0.001 to 10% (wgt/vol). Particularly preferred detergent solution contains TRITON X-100™ which is chemically (polyethylene glycol P-1,1,3,3-tetramethylbutylphenyl ether, octyl phenol ethoxylate, 4-octylphenol polyethoxylate, Mono 30; Molecular formula: C₁₄H₂₂O (C₂H₄O)ₙ where the average number of ethylene oxide units per molecule is around 9 or 10) at a concentration between 0.01 to 1% wgt/volume.

In a most preferred embodiment, the sampling swab 27 is loaded with from 40 to 80 μL (preferably 75 μL) of a wetting solution, preferably 1 to about 10% propanol and about 1% detergent. The sampling swab 27 is of a size and composition such that the maximum loading of the swab 27 would be approximately 100-200 μL of the solution with the BCA reagent from the reservoir 23. With a preferred silicone elastomer composition, the cylindrically shaped sampling swab 27 would be approximately 8 mm in diameter and 1.5-1.8 mm in height (dry or pre-wet).

It should be noted that, according to the present invention, the exact loadings and capacity of the sampling swab 27 are not absolute. What is important to the practice of the methods of the present invention is that the sampling swab, whatever its specific geometry, or its absolute capacity to absorb and hold a solution of an extracting agent, be loaded with a solution of such agent to a level that is somewhat below the saturation capacity of the swab material. The specific significance of this loading level will be addressed in more detail below.

As can be seen from the Figures, including FIG. 6, the sampling swab 27 is represented as having a regular cylindrical geometry. As should also be apparent to one of skill in the appropriate art, the use of a regular cylindrical geometry is for illustrative purposes only, and is not intended to limit the range of suitable geometries for the sampling swab 27 in the practice of the present invention. For example, it may prove to be advantageous to provide the sampling swab 27 in a geometry where the bottom surface of the swab cylinder that actually comes in contact with the surface to be sampled is not parallel to the top surface of the swab. In this regard, the bottom surface of the sampling swab 27 is angled downward. Thus configured, the sampling swab may be better able to reach less accessible portions of the surface to be sampled, such as corners or ridges or other surface irregularities, particularly where that surface is not perfectly planar and/or regular.

Once the sampling swab 17 has been used to collect a sample from the surface onto the sampling swab 27, the sampling wand 17 is returned to the sampling/analysis member 15 where the wand is re-inserted into the inner chamber 40 of the sampling/analysis member. When first re-inserted, the sampling wand 17 can be returned to its original longitudinal position within the inner chamber 40 of the sampling/analysis member 15. In that position, the member 15 is in substantially the same arrangement as depicted in FIG. 5. In that arrangement, upper seal 29 remains undisturbed, and the contents of the reservoir 23 are intact.

FIG. 7 illustrates the sampling wand 17 moved longitudinally within the inner chamber 40 of the sampling/analysis member to a first operational position. In this first operational position, the sampling wand 17, has been moved downward so that the point 52 of the piercing member 53 moves upward, in a relative sense, piercing the sampling swab 27, upper seal 29, and releasing the contents of the reagent solution from the reservoir 23. The reagent solution thus released travels downward out of the reservoir 23 and diffuses through the sampling swab 27 and into the distal end of the inner chamber 40 of the sampling/analysis member 15. As the solution diffuses through the sampling swab 27, it effectively rinses the sample obtained from the surface to be analyzed into the solution collected at the bottom of the inner chamber.

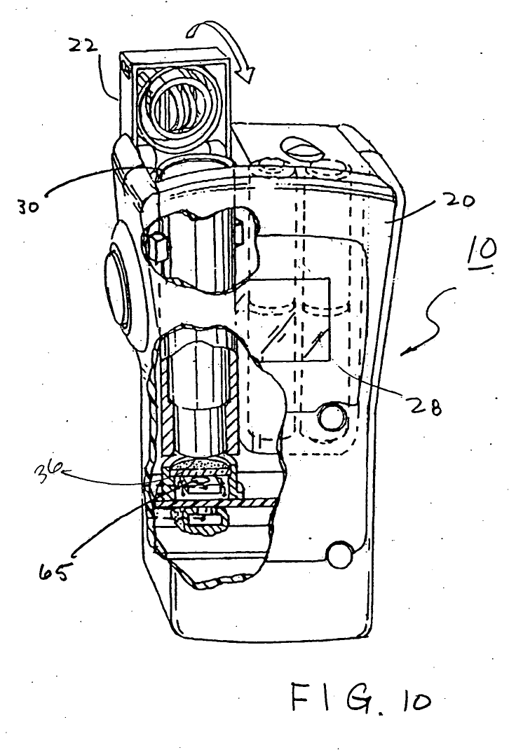

From this first operational position, the sampling wand 17 may be urged further downward to a second operational position, as shown in FIG. 8, and in magnified detail in FIG. 9. In so moving, the cutting edge 58 of the cutting member 56 is forced to break through the first and, if present, second seals, 49 and 50, respectively. In doing so, the reagent solution from the reservoir, that has effectively removed from the sampling swab 27 any protein from the surface to be analyzed, is permitted to flow further downward through the inner chamber 40 and into the reaction well 36 at the distal end of the outer chamber 55. In returning the sampling wand 17 to the sampling/analysis member 15, and moving the sampling wand downward to the first and second operational positions, the outer surface of the outer chamber 55 or of the sampling wand 17 may be provided with external markings, such as circumferential rings, indicating to the operator the appropriate positions to which to move the distal end of the sampling wand 17. As described immediately above, the sampling wand 17 may be moved downward through the chambers 40 and 55 of the sampling/analysis member 15 in a step-wise progression. However, it is also possible to move the sampling swab downward in a single movement without pausing between the first and second operational positions.

As the bicinchoninic acid or salt compound solution moves to the reaction well of the outer chamber 55, the solution comes into contact with the disc 48 containing the dried cupric salt. As a result of this contact, the cupric salt contained therein is rehydrated and reduced to a cuprous (Cu⁺¹) ion by the protein. The cuprous ion reacts with the bicinchoninic acid or salt to produce a visible color (purple complex) which is in contrast to the light green color of the bicinchoninic compound in solution. In one preferred embodiment, the color is more visually delivered. It can also be detected by a spectrophotometer device 10 as shown in FIG. 1. This is valuable for faint color changes.

Using techniques known to one of ordinary skill in the appropriate electronics arts, it is possible to design the detector and display circuitry of the spectrophotometer 20 to process the output signal so as to report an optimized reading obtained most likely in that a 10 to 15 minute time window of the reaction (at ambient temperatures).

By reference to FIG. 10, it is possible to see, via the cutout views in the Figure, the position of the reaction well and, more specifically, the disc cavity 45, relative to the detector circuitry 65. The bottom wall of the reagent disc cavity 45 is transparent so that color from the reaction taking place within the reaction well 36 is deterred by a monochromatic light beam which reaches a spectrophotometer detector 65. Light at 562-600 nm can be impinged on the clear cavity 45 and then be detected by a photodiode. Alternatively a grayscale photographic sensor or CCD camera could be used.

Referring back now to the individual components of the sampling/analysis member 15, it is useful to note certain characteristics and operational specifications of these components. Turning first to the sampling swab 27, successful and optimal practice of the present invention places certain requirements on the material used for the swab 27. As can be seen from the discussion of the prior art provided above, the vast majority of the prior art sampling and/or analysis devices disclosed therein utilize a “Q-Tip®” type sampling swab. As such, the swabs of the prior art were composed primarily of cotton or other fibrous materials, whether natural or man-made, or a combination thereof. Although such materials can be utilized in a variety of applications, the present inventors have determined that practice of the present invention can be optimized through selection of the proper material for use as the sampling swab 27. Toward this end, the preferred material for use as the sampling swab is of an inorganic silicone elastomer polymeric in nature, as opposed to the natural or polymer fibrous material that predominates in the prior art. Use of a silicone elastomer material provides a number of advantages (e.g. chemically inert, heat resistant, chemical stability, ability to release organic compound easily to transfer protein) in the fabrication of the swab and also its incorporation into the sampling wand 17. The preferred geometry for the sampling swab 27 of the present invention provides a flat surface that maximizes surface area contact between the swab and the surface to be analyzed. Use of the preferred alternative geometry wherein the bottom surface of the swab is not parallel to the top surface further increases effective sampling surface area for the swab for a given cylindrical radius, but also provides a relatively sharp edge that can be effective in reaching irregularities in the sampled surface. A further advantage of an appropriate silicone elastomer is that it can be sterilized by chemical means. This is a characteristic that is essential given the primary uses of the device of the present invention.

Use of a silicone elastomer material for the sampling swab 27 makes it possible to select and control optimal physical and chemical properties of the swab that enhance the effectiveness of the practice of the present invention. For example, as discussed above, the sampling swab 27 is pre-wetted, preferably with an extractant solution. It is important to effective sampling of a surface to be analyzed that the sampling swab be pre-wetted with solution at a loading that is somewhat below the saturation capacity of the swab material. With a silicone elastomer material as the sampling swab, it is possible to fabricate the swab with specific densities and internal pore sizes so as to be able to achieve specific fluid loading characteristics, and to insure that these characteristics are met uniformly both throughout the swab and also from one swab to the next.

Chemically speaking, the silicone material is highly resistant to chemical attack, including attack from fluids with both high and low pH (basic and acidic, respectively). Thus, the film is an extremely durable material. This is particularly advantageous in a component of the device that may have a relatively long expected shelf life. The mechanical durability of the swab is also superior to prior art swabbing materials. As will be illustrated below, an important characteristic of the preferred material for the sampling swab 27 is that the absorbent nature of the material provides nearly instantaneous wicking when in contact with moisture. This greatly facilitates the sampling process, described immediately below, whereby bacterial organisms are removed from a surface to be sampled.

The inventors have determined that a particularly preferred type of polymeric material for the disc 48 is composed of the reaction product of polyvinyl alcohol and an aldehyde. Porous silicone elastomer can also be used. In this regard, reference is made to U.S. Pat. No. 4,098,728, the disclosure of which, herein incorporated specifically by reference, teaches methods for the preparation of such polymeric species. However, based on the disclosure contained herein, one of skill in the appropriate art will recognize that organic polymeric materials will serve as well, provided these materials possess the desired physical and chemical properties.

In actual use, as illustrated in FIG. 6, the sampling wand 17, after extraction from the sampling/analysis member 15, is positioned above the surface to be analyzed so that the sampling swab 27 at the distal end of the sampling wand 17 is in contact with the surface. In a preferred method of use, the pre-wetted sampling swab is pressed to the surface to be analyzed with sufficient force that, as the sampling swab 27 is moved across the surface, the extractant solution with which it has been pre-wetted is expelled from the swab and spread over the surface to be analyzed. The sampling wand 17 is then wiped again across the now wet surface. The absorptive capacity of the silicone elastomer is such that the swab effectively reabsorbs the moisture from the surface to be sampled.

Although the Figures and the description provided above are primarily directed to the use of the device and methods of the present invention in the sampling of solid surfaces, it should be noted that the device and methods disclosed herein are particularly suited to adaptation for use with other types of samples and alternative methodology. For example, the device of the present invention can readily be used to sample for materials indicative of the presence of proteins in liquid samples and not just on solid surfaces. To obtain a sample from a liquid source using the sampling wand 17 of the present invention, the swab 27 on the sampling wand should contain an effective amount of an extracting agent such as a detergent. The swab 17 can be loaded with a detergent solution simply by contacting the swab to an appropriate solution. Alternatively, the swab can be further treated after contacting a detergent solution by evaporation of the solvent from the detergent solution, leaving behind the solute detergent species. The specific characteristics of the silicone material of which the swab is comprised are particularly well suited for this practice due to the large void volume within the silicone and the resulting absorptive capacity of the swab. Furthermore, the large internal surface area within the silicone material arising from the large void volume provides optimal conditions for the rapid mixing of liquids with the dry reagents, such as a detergent, loaded into the swab.

When sampling, a liquid, the sampling wand 17 can simply be contacted with the liquid, and the high absorptive capacity of the swab 27 should result in an almost instantaneous wicking of the liquid to be sampled into the swab. Alternatively, the liquid to be sampled can be transferred directly to the swab 27 by a dropper, pipette, or other suitable transfer means. If necessary to acquire a sample of sufficient volume, the size of the sampling swab 27 can be increased. Because it is important for the swab material to retain capacity to absorb additional fluid when sampling a liquid, it is necessary to avoid pre-wetting the swab 27 to absorptive saturation or the swab will be unable to retain a sufficient volume of the sampled liquid. Therefore, care must be taken when wetting the swab 27 when it is the intention of the operator to use the swab in a pre-moistened state. It can be preferable, then, to utilize the swab 27 where the solvent from the detergent solution is evaporated away.

organic analytes such as proteins and sugars are preferentially retained on the surface of the swab by hydrophobic interaction, yet they can be easily delivered to the reaction well by washing. It should be recognized that one of the potential problems associated with sampling liquids is that the protein may not be present at sufficiently high concentration levels to provide a meaningful sample. This situation is not unusual when assaying a liquid sample. However, it is possible to pre-concentrate the protein in the liquid by filtering the liquid through an appropriate filter, such as one with a filter size of approximately 0.2 microns (μm). After the filtering step, the sampling wand 17 can be swiped across the surface of the filtering medium to acquire the concentrated sample. The sampling wand can then be used in a manner consistent with the sampling of solid surfaces, as described above.

### EXAMPLE 1

A test device as shown in FIGS. 1 to 10 made of polyvinyl alcohol polymer was provided with a disc 48 (Merocel, Mystic, Conn.) was filled with a Reagent A and then dried. Composition A was (1% CUSO₄.5H₂O) with 1% TRITON X-100™, 10 μL of this solution was placed on the polyvinyl alcohol disk 1 mm×4 mm diameter and dried in a vacuum oven at 50-60° C. for 30-60 minutes.

The reservoir 23 in sampling wand 17 was filled with Composition B. Composition B was an aqueous solution of 1% bicinchoninic acid-sodium salt (Pierce Chemical Company (Rockford, Ill.), 2% Na₂CO₃.H₂O, 0.16% disodium tartrate, 0.4% NaOH and 0.95% NaHCO₃ at pH 11). 200 uL of this solution was placed in the reservoir 23 of the wand 17. All salts and buffer substances were of analytical grade.

The swab 27 was a silicone SILASTIC® from (Dow Corning, Midland, Mich.) with methyl or lower alkyl groups (1 to 6 carbon atoms) as side chains on the silicone. The seals 29 and 50 were polypolypropylene with a thermally activated adhesive on one surface (AET Films (Newcastle, Del.). The polyolefin films do not interfere with the bicinchoninic acid or salt reaction. Metal films such as aluminum are not suitable since they reduce Cu⁺² to Cu⁺¹. The swab was a Mylar backed porous silicone elastomer. The Mylar was secured by a silicone adhesive to the silicone polymer and to the polypropylene film.

In tests the swab 17 was removed from the analysis structure or container 30. The swab 27 contained a 10% by volume ethanol and 1% by volume TRITON X-100™ aqueous solution. The test used bovine serum albumin (BSA) as the protein on a surface. The reaction was conducted for 10 minutes. The color intensity was determined using an Eastman Kodak (Rochester, N.Y.) 1D densitometer program. The color was also observed visually. The result was that the test device functioned very well and generated on transparent disc cavity 45 a deep purple color (at 562 nm wavelength) characteristic of the cuprous ion bicinchoninic acid or salt complex. The test device was able to detect down to 30 μg of BSA visually. In further tests in the field the test device was able to detect protein (unsanitary conditions) even with minute amounts of protein contamination. The control reaction without protein produced the characteristic light green color of copper sulfate (470 nm).

### EXAMPLE 2

Using the spectophotometric assay device 10 of FIG. 1, the purple cuprous bicinchoninic protein complex could be detected with a light beam at 562 nm through a side of the cavity 45.

It is preferred that the wand 17 be shaken three (3) times after it is inserted into the analysis structure 30 to insure that liquid in the reservoir 23 flows through the swab 27 and into the disc cavity. It is also noted that the cutting member 56 and second seals 49 and 50 are unnecessary functionally, although for manufacturing purposes they are useful. It is only necessary that the piercing member 53 penetrate the swab 27 and first seal 29.

It is intended that the foregoing description be only illustrative of the present invention and that the present invention be limited only by the hereinafter appended claims.

We claim:

1. A test device comprising:
(a) a sampling wand for an assay for the presence of protein or other reducing compound on a surface, the wand comprising:
(i) an internal reservoir disposed toward a distal end of the wand;
(ii) a bicinchoninic acid or alkali metal salt compound in solution within the internal reservoir;
(iii) an external sampling swab disposed on a surface at the distal end of the wand for picking up protein on the surface; and
(iv) a frangible seal disposed between the sampling swab and the internal reservoir, so that when the seal is broken the solution containing the compound rinses through and from the swab to release any protein from the swab; and
(b) a container for inserting the wand and for receiving the released solution from the wand so that in the presence of any of the protein or other reducing compound a cupric ion from a dried inorganic cupric salt in the container is reduced to a cuprous ion by the protein or other reducing compound and reacts with the compound to produce a color indicating the presence of the protein or other reducing compound.

2. The test device of claim 1 wherein the cupric salt is dried in the presence of a non-ionic surfactant on a porous member in the container.

3. The test device of claim 1 wherein the cupric salt is cupric sulfate pentahydrate dried with a non-ionic surfactant on a porous member, which is comprised of a silicone elastomer, in a well in a bottom portion of the container.

4. The test device of claim 3 wherein the porous member is a polyvinyl alcohol.

5. The test device of claim 3 or 4 wherein the cupric sulfate pentahydrate was provided by a 1% by weight to volume aqueous solution of the cupric sulfate hexahydrate with 0.05% by volume non-ionic surfactant which is dried on the porous member.

6. The test device of claims 2 or 3 wherein the non-ionic surfactant is TRITON X-100™ (polyethylene glycol tert-octylphenyl ether).

7. The test device of claim 1 wherein the sampling swab on the wand is comprised of a porous, absorbent material.

8. The test device of claim 4 wherein the sampling swab has a cylindrical shape.

9. The test device of claim 1 wherein the sampling swab has a height which is less than a diameter and is comprised of a porous silicone elastomer.

10. The test device of claim 1 further comprising a non-ionic surfactant as an extracting agent absorbed in the sampling swab.

11. The test device of claim 10 wherein the non-ionic surfactant is TRITON X-100™ (polyethylene glycol tert-octylphenyl ether).

12. The test device of claim 1 wherein the solution in the wand is a buffered solution at a basic pH.

13. The test device of claim 1 wherein the composition is a sodium salt and has a concentration of 0.01 to 1% (wt/vol) in the solution in the wand which is buffered with citrate and tartarate buffer and is at a pH of about 11.

14. The sampling wand of claim 1 wherein the solution in the wand comprises a buffer to dissolve compound BCA and is at a basic pH.

15. The test device of claim 1 wherein a puncturing means for the frangible seal is provided in the container so that when the wand is inserted into the container the frangible seal is punctured by a piercing member to release the solution from the wand, through the swab and into the container with the cupric salt.

16. The test device of claim 5 wherein the swab and the frangible seal are both punctured.

17. A method for detecting a protein or other reducing compound on a surface which comprises:
(a) providing a test device comprising:
(i) a sampling wand for an assay for the presence of protein or other reducing compound on a surface, the wand comprising:
(ii) an internal reservoir disposed toward a distal end of the wand;
(iii) a bicinchoninic acid or alkali metal salt compound in solution within the internal reservoir;
(iv) an external sampling swab disposed on a surface at the distal end of the wand for picking up protein or other reducing compound on the surface; and
(iv) a frangible seal disposed between the sampling swab and the internal reservoir, so that when the seal is broken the solution containing the compound rinses through and from the swab to release any of the protein or other reducing compound from the swab; and a container for inserting the wand and for receiving the solution from the wand so that in the presence of protein a cupric ion from a dried inorganic cupric salt in the container is reduced to a cuprous ion by the protein and reacts with the compound to produce a color indicating the presence of the protein;
(b) wiping the sampling swab on the wand over a surface to pick up any protein or reducing compound;
(c) inserting the wand in the container and breaking the frangible seal so that the released solution washes the swab contacts the inorganic cupric salt and any protein or reducing compound reduced the cupric ion to the cuprous ion and reacts with the compound (BCA); and
(d) determining whether or not the protein or other reducing compound was present on the surface, wherein green is negative and purple is positive for the protein or other reducing compound.

18. The method of claim 17 wherein the test device is provided with a puncturing means for the frangible seal in the container and wherein the wand is inserted into the container, the frangible seal is broken by the puncturing means to release the solution from the wand through the swab and into the container with the cupric salt.

19. The method of claim 18 wherein both the frangible seal and the swab are punctured.

20. The method of claim 17 wherein the cupric salt is dried with a non-ionic surfactant on a porous member in the container.

21. The method of claim 17 wherein the cupric salt is cupric sulfate pentahydrate dried with a non-ionic surfactant on a porous member, which is comprised of a silicone elastomer, in a well in a bottom portion of the container.

22. The method of claim 17 wherein the porous member is a polyvinyl alcohol.

23. The method of any one of claim 21 wherein the cupric sulfate pentahydrate was provided by a 0.05% by volume to volume aqueous solution of the cupric sulfate hexahydrate and a 1% by weight non-ionic surfactant which is dried on the porous member.

24. The method of claim 21 wherein the non-ionic surfactant is TRITON X-100™ (polyethylene glycol tert-octylphenyl ether).

25. The method of claim 17 wherein the sampling swab on the wand is comprised of a porous, absorbent material.

26. The method of claim 17 wherein the sampling swab has a cylindrical shape.

27. The method of claim 17 wherein the sampling swab has a height which is less than a diameter and is comprised of a silicone elastomer.

28. The method of claim 27 further comprising a non-ionic surfactant as an extracting agent absorbed in the sampling swab.

29. The method of claim 28 wherein the non-ionic surfactant is TRITON X-100.

30. The method of claim 17 wherein the solution in the wand is a buffered solution at a basic pH.

31. The method of claim 17 wherein the compound is as a sodium salt and has a concentration of 0.01-1% (wt/vol) in the solution in the wand which is buffered with citrate or tartaric buffer and is at a pH of about 12.

32. The sampling wand of claim 17 wherein the solution in the wand comprises an acid neutralizing agent and is at a basic pH.
